(19) United States
(12) Patent Application Publication
(10) Pub. No.: US 2006/0018865 A1
(43) Pub. Date: Jan. 26, 2006

(54) Foaming cleansing composition

(75) Inventor: Odile Aubrun-Sonneville, Antony (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/183,797

(22) Filed: Jul. 19, 2005

Related U.S. Application Data
(60) Provisional application No. 60/592,323, filed on Jul. 30, 2004.

(30) Foreign Application Priority Data
Jul. 26, 2004 (FR) 0451652

(51) Int. Cl. A61K 8/55; A61K 8/36

(52) U.S. Cl. 424/70.22; 424/70.23

(57) ABSTRACT

A cleansing composition for topical application containing at least one surfactant chosen from alkyl glycol carboxylic acids and their salts, and at least one surfactant chosen from monoalkyl phosphates, dialkyl phosphates and their salts. The composition has good cosmetic properties, and the foam obtained is fine and rinses off well.

### REFERENCE TO PRIOR APPLICATIONS

This application claims priority to U.S. provisional application 60/592,323 filed Jul. 30, 2004, and to French patent application 0451652 filed Jul. 26, 2004, both incorporated herein by reference.

### FIELD OF THE INVENTION

The invention relates to a composition comprising a surfactant system containing at least one carboxylic ionic surfactant and at least one surfactant of the phosphate type. Preferably the invention composition is provided in the form of a foaming cleansing composition which can be rinsed off with water and which has the appearance of a gel. Also described is the use of the invention composition in the cosmetics and dermatological fields, in particular as a product for cleansing or removing makeup from the skin, scalp and/or the hair.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Cleansing the skin is very important for caring for the face. It must be as efficient as possible since greasy residues, such as excess sebum, the remnants of cosmetic products used daily, and makeup products, in particular waterproof products, accumulate in the skin folds and can block the pores of the skin and result in the appearance of spots.

The use of foaming detergent aqueous gels for cleansing the skin is known. Their cleansing action is provided by the surfactants that they comprise, these surfactants suspending the greasy residues and the pigments of the makeup products. These gels are effective and pleasant to use because they give a good foam. When a foaming gel is used, the aim is to have a gel that gives good foam qualities, i.e. an abundant foam that forms readily and rapidly, has fine bubbles and exhibits good hold (does not run), and that is rapidly rinsed off without leaving any residual film. In addition, comfortable gels are increasingly being sought, i.e. gels that leave a comfortable feeling on the skin after use, and which therefore result neither in tightness nor in feelings of dryness of the skin.

Soaps (fatty acid salts) are generally used to obtain a very good foam quality. Compositions containing soaps generally contain a very high soap content (20-30%), which contributes both to the good foaming performance levels and to the texture quality. However, compositions containing soaps are less well tolerated, particularly by irritable skin, since they produce a feeling of dryness and can be irritant.

Other foaming products are obtained with ionic or non-ionic synthetic surfactants, such as sodium lauryl ether sulphate, alkylbetaines such as laurylbetaine, or alkylpolyglucosides (APGs). The foams that they generate are generally coarse and exhibit a poor hold since they run instead of holding well on the skin. A new surfactant family, the alkyl glycol carboxylates, produces foams that are more dense but nevertheless coarse.

There therefore remains a need for producing gels that are both foaming and gentle and that exhibit very good foam quality.

### SUMMARY OF THE INVENTION

The inventor has discovered, surprisingly, that a foaming gel having both good cosmetic properties (e.g., qualities of the foam and quality of the rinsing) and good properties of tolerance are provided by using a surfactant system comprising at least one alkyl glycol carboxylate and at least one surfactant chosen from mono-alkyl phosphates, dialkyl phosphates and their salts.

### DETAILED DESCRIPTION OF THE PREFERRED EMBODIMENTS

One subject of the present application is therefore a cleansing composition for topical application, comprising, in a physiologically acceptable aqueous medium, a surfactant system containing at least one surfactant chosen from alkyl glycol carboxylic acids and their salts, and at least one surfactant chosen from monoalkyl phosphates, dialkyl phosphates and their salts.

The term “topical application” is here intended to mean an external application to the keratin materials, namely in particular the skin, the scalp, the eyelashes, the eyebrows, the nails, the hair and/or the mucous membranes. Since the composition is preferably intended for topical application, it preferably comprises a physiologically acceptable medium. Moreover, the term “physiologically acceptable medium” is intended to mean a medium that is compatible with the skin, the lips, the scalp, the eyelashes, the eyes, the nails and/or the hair. The composition may in a preferred embodiment constitute a cosmetic or dermatological composition.

Moreover, the term “aqueous medium” is intended to mean a medium containing an amount of water of at least 35% by weight, preferably ranging from 35 to 98% by weight, and better still from 40 to 80% by weight, relative to the total weight of the composition. The aqueous medium of the foaming compositions of the invention can comprise, for example besides water, one or more solvents chosen from lower alcohols containing from 1 to 6 carbon atoms, such as ethanol; polyols such as glycerol, glycols such as butylene glycol, isoprene glycol, propylene glycol and polyethylene glycols such as PEG-8, sorbitol; sugars such as glucose, fructose, maltose, lactose or sucrose; and mixtures thereof. The amount of solvent(s) in the composition of the invention can range from 0.5 to 30% by weight, and preferably from 5 to 20% by weight, relative to the total weight of the composition.

The compositions of the invention are preferably foaming cleansing compositions that can be rinsed off, and that can be used in the skin, hair or mucous membrane cleansing field.

The composition is preferably in the form of a fluid or of a gel. The viscosity of the compositions according to the invention preferably ranges from 0.01 to 50 Pa.s, measured at 25° C. using the Rheomat RM180 from Rheometric Scientific at 200 rpm (revolutions per minute), 10 minutes after the rotation of the rotor has begun. The device is equipped with a different rotor according to the viscosities, for example with a rotor 2 for ranges of viscosities of less than 0.7 Pa.s, with a rotor 3 for ranges of viscosities of 0.2 to 4 Pa.s, and with a rotor 4 for ranges of viscosities of greater than 2 Pa.s.

The compositions of the invention have the advantage of being very stable and of not exhibiting any phase separation or phenomena of recrystallization when stored at from 4° C. to 45° C.

The surfactant system in the composition according to the invention may be present in an amount (in terms of active material) ranging for example from 2 to 30% by weight, preferably from 3 to 20% by weight, relative to the total weight of the composition.

The weight ratio of the surfactants chosen from alkyl glycol carboxylic acids and their salts to the surfactants chosen from monoalkyl phosphates, dialkyl phosphates and their salts can range for example from 90/10 to 10/90, and preferably from 25/75 to 75/25.

Alkyl Glycol Carboxylic Acids

The composition according to the invention comprises at least one surfactant chosen from alkyl glycol carboxylic acids (or 2-(2-hydroxyalkyloxyacetic) acids) their salts, and their mixtures. These are anionic surfactants.

These surfactants can in particular have the formula (I) below:
R₁—CHOH—CH₂—O—CH₂—COO⁻X⁺ (I)
in which R₁ denotes a linear or branched, saturated or unsaturated alkyl radical containing from 8 to 30 carbon atoms, and X denotes hydrogen or a mineral or organic cation such as those derived from an alkali metal (for example Na+, K+), NH₄+, ammoniums derived from basic amino acids such as lysine, arginine, sarcosine, ornithine or citrulline, or else amino alcohols such as monoethanolamine, diethanolamine, triethanolamine, glucamine, N-methylglucamine or 3-amino-1,2-propanediol.

The 2-hydroxyalkyl carboxylic acids that are preferred according to the present invention are compounds of formula (I) in which R₁ denotes more particularly a linear or branched, saturated or unsaturated alkyl radical containing from 8 to 18 carbon atoms.

Among the surfactants of formula (I), mention may in particular be made of sodium lauryl glycol carboxylate, sold under the names Beaulight Shaa® or Beaulight LCA-25N® by the company Sanyo, or its corresponding acid form sold under the name Beaulight Shaa (Acid Form)® sold by the company Sanyo.

The amount of surfactants of alkyl glycol carboxylic type can range for example from 1 to 20% by weight (in terms of active material), preferably from 1 to 15% by weight, relative to the total weight of the final composition.

Monoalkyl Phosphates and Dialkyl Phosphates

The composition according to the invention comprises at least one surfactant chosen from monoalkyl phosphates, dialkyl phosphates, their salts and their mixtures. These are also anionic surfactants.

These monoalkyl phosphates and dialkyl phosphates contain one or more linear or branched, aliphatic and/or aromatic alkyl chains having from 8 to 22 carbon atoms. In the mixture of monoalkyl and dialkyl phosphates, the mono/di proportion can range from 100/0 to 50/50. These phosphates can be neutralized with organic or inorganic bases such as potassium hydroxide, sodium hydroxide, triethanolamine, arginine, lysine and N-methylglucamine.

As surfactants of the phosphate type that can be used in the composition of the invention, particular mention may, for example, be made of lauryl monophosphate, such as the product sold under the name MAP 20® by the company Kao Chemicals, the potassium salt of dodecylphosphoric acid, such as the mixture of mono- and diester (predominantly diester) sold under the name Crafol AP-31® by the company Cognis, the monoester and diester of octylphosphoric acid, such as the mixture sold under the name Crafol AP-20® by the company Cognis, the monoester and diester of ethoxylated (7 mol of EO) 2-butyloctanyl phosphoric acid, such as the mixture sold under the name Isofol 12 7 EO-Phosphate Ester® by the company Condea, the potassium or triethanolamine salts of (C₁₂-C₁₃) monoalkyl phosphate, such as the product sold under the references Arlatone Map 230K-40® and Arlatone Map 230T-60® by the company Uniqema, potassium lauryl phosphate, such as the product in 40% aqueous solution sold under the name Dermalcare Map XC-99/09® by the company Rhodia Chimie, and mixtures of these surfactants.

The amount of surfactant(s) of the phosphate type can range, for example, from 0.5 to 10% by weight of active material, and preferably from 0.5 to 7% by weight of active material, relative to the total weight of the composition.

Additional Surfactants

The surfactant system can contain, besides the surfactants mentioned above, one or more other additional surfactants. However, these other surfactants, if they are present, should be in an amount of less than 50% by weight relative to the total amount of surfactant system. These surfactants can be present, for example, in an amount (in terms of active material) ranging from 0.5 to 10% by weight, and preferably from 1 to 7% by weight, relative to the total weight of the composition.

These additional surfactants can be chosen from non-ionic, amphoteric and anionic surfactants, and mixtures thereof, for example.

Non-ionic Surfactants

As non-ionic surfactants, use may, for example, be made of alkylpolyglucosides (APG), maltose esters, polyglycerolated fatty alcohols, glucamine derivatives such as 2-ethylhexyloxycarbonyl-N-methyl-glucamine, and mixtures thereof.

As alkylpolyglucosides, use is preferably made of those comprising an alkyl group containing from 6 to 30 carbon atoms, and preferably from 8 to 16 carbon atoms, and comprising a hydrophilic (glucoside) group preferably comprising 1.2 to 3 saccharide units. As alkylpolyglucosides, mention may, for example, be made of decylglucoside ((C₉/C₁₁)alkylpolyglucoside (1.4)), such as the product sold under the name Mydol 10® by the company Kao Chemicals, the product sold under the name Plantaren 2000 UP® by the company Cognis and the product sold under the name Oramix NS 10® by the company Seppic; caprylyl/capryl glucoside, such as the product sold under the name Oramix CG 110® by the company Seppic; laurylglucoside such as the products sold under the names Plantaren 1200 N® and Plantacare 1200® by the company Cognis; and cocoglucoside such as the product sold under the name Plantacare 818/UP® by the company Cognis.

The maltose derivatives are, for example, those described in document EP-A-566438, such as O-octahoyl-6′-D-maltose, or else O-dodecanoyl-6′-D-maltose described in document FR-2,739,556.

Among the polyglycerolated fatty alcohols, mention may be made of polyglycerolated dodecanediol (3.5 mol of glycerol), a product sold under the name Chimexane NF® by the company Chimex.

Amphoteric Surfactants

The amphoteric and zwitterionic surfactants can be chosen, for example, from derivatives of betaines, amphoacetates, hydroxysultaines, and mixtures thereof.

As betaines, mention may, for example, be made of cocobetaine, such as the product sold under the name Dehyton AB-30® by the company Cognis, laurylbetaine, such as the product sold under the name Genagen KB® by the company Clariant, oxyethylenated (10 EO) laurylbetaine, such as the product sold under the name Laurylether(10 EO)betaine® by the company Shin Nihon Rioca, and oxyethylenated (10 EO) stearylbetaine, such as the product sold under the name Stearylethyl(10 EO)betaine® by the company Shin Nihon Rica.

As alkyl amphoacetates, mention may, for example, be made of disodium N-cocoyl-N-carboxy-methoxyethyl-N-carboxymethyl-N-ethylenediamine (CTFA name: disodium cocamphodiacetate) such as the product sold under the name Miranol C2M Concentré NP® by the company Rhodia Chimie, and sodium N-cocoyl-N-hydroxyethyl-N-carboxymethyl-N-ethylenediamine (CTFA name: sodium cocamphoacetate).

As hydroxysultaines, mention may be made of coamidopropyl hydroxysultaine such as the product sold under the name Rewoteric AM CAS by the company Goldschmidt-Degussa.

Anionic Surfactants:

As anionic surfactants, use may, for example, be made of carboxylates, alkyl sulphates, sulphonates, sulphosuccinates, alkyl sulphoacetates, phosphates, polypeptides, anionic derivatives of alkylpolyglucosides, and mixtures thereof.

As carboxylates, mention may be made of:
amido ether carboxylates (AECs), such as sodium lauryl amido ether carboxylate (3 EO), sold under the name Akypo Foam 30® by the company Kao Chemicals;
polyoxyethylenated carboxylic acid salts, such as oxyethylenated (6 EO) sodium lauryl ether carboxylate (C₁₂₋₁₄₋₁₆ 65/25/10) sold under the name Akypo Soft 45 NV® by the company Kao Chemicals, polyoxyethylenated and carboxymethylated fatty acids of olive oil-origin sold under the name Olivem 400® by the company Biologia E Tecnologia, oxyethylenated (6 EO) sodium tridecyl ether carboxylate sold under the name Nikkol ECTD-6NEX® by the company Nikkol;
alkali metal salts of N-acylamino acids;
sarcosinates, such as sodium lauroyl sarcosinate sold under the name Sarkosyl NL 97® by the company Ciba or sold under the name Oramix L 30® by the company Seppic, sodium myristoyl sarcosinate sold under the name Nikkol Sarcosinate MN® by the company Nikkol, sodium palmitoyl sarcosinate sold under the name Nikkol Sarcosinate PN® by the company Nikkol;
alaninates, such as sodium N-lauroyl-N-methyl amidopropionate sold under the name Sodium Nikkol Alaninate LN30® by the company Nikkol, or sold under the name Alanone ALE® by the company Kawaken, N-lauroyl-N-methylalanine triethanolamine sold under the name Alanone ALTA® by the company Kawaken;
glutamates, such as triethanolamine monococoylglutamate sold under the name Acylglutamate CT-12® by the company Ajinomoto, triethanolamine lauroylglutamate sold under the name Acylglutamate LT-12® by the company Ajinomoto;
aspartates, such as the mixture of triethanolamine N-lauroylaspartate/triethanolamine N-myristoylaspartate sold under the name Asparack® by the company Mitsubishi;
glycinates, such as sodium N-cocoylglycinate sold under the names Amilite GCS-12® and Amilite GCK 12 by the company Ajinomoto;
citrates such as the citric monoester of oxyethylenated (9 mol) coco alcohols, sold under the name Witconol EC 1129 by the company Goldschmidt;
galacturonates such as sodium dodecyl d-galactoside uronate sold by the company Soliance;
fatty acid salts (soaps) having a C₆-C₂₂ alkyl chain, neutralized with an organic or mineral base such as potassium hydroxide, sodium hydroxide, triethanolamine, N-methylglucamine, lysine or arginine.

As alkyl sulphates that may or may not be oxyethylenated, mention may, for example, be made of sodium lauryl ether sulphate (C₁₂-C₁₄ 70/30) (2.2 EO) sold under the name Sipon AOS 225® by the company Cognis, ammonium lauryl ether sulphate (C₁₂-C₁₄ 70/30) (3 EO) sold under the name Sipon LEA 370® by the company Cognis, ammonium. (C₁₂-C₁₄)alkyl ether (9 EO) sulphate sold under the name Rhodapex AB/20® by the company Rhodia Chimie, the mixture of sodium lauryl- and oleyl-ether sulphate and magnesium lauryl- and oleyl-ether sulphate sold under the name Empicol BSD 52 by the company Albright & Wilson.

As sulphonates, mention may be made, for example, of:

alpha olefin sulphonates such as sodium alpha olefin sulphonate (C₁₄₋₁₆) sold under the names Bio-Terge AS-40® and Bio-Terge AS-40 CG® by the company Stepan, or sold under the name Witconate AOS Protege®, Sulframine AOS PH 12® sold by the company Witco, the secondary sodium olefin sulphonate sold under the name Hostapur SAS 30® by the company Clariant;
isethionates such as sodium cocoyl isethionate sold under the name Jordapon CI P® by the company Jordan;
taurates such as the sodium salt of palm kernel oil methyltaurate sold under the name Hostapon CT Pate® by the company Clariant, sodium N-cocoyl-N-methyl-taurate sold under the name Hostapon LT-SF® by the company Clariant or Nikkol CMT-30-T® by the company Nikkol, or sodium palmitoyl methyltaurate sold under the name Nikkol PMT® by the company Nikkol.

As sulphosuccinates, mention may, for example, be made of oxyethylenated (3 EO) lauryl (C₁₂-C₁₄ 70/30) alcohol monosulphosuccinate sold under the name Setacin 103 Special® by the company Zschimmer Schwarz, or sold under the name Rewopol SB-FA 30 K 4® by the company Witco, the disodium salt of a hemi-sulphosuccinate of C₁₂-C₁₄ alcohols sold under the name Setacin F Special Paste® by the company Zschimmer Schwarz, oxyethylenated (2 EO) disodium oleamido sulphosuccinate sold under the name Standapol SH 135® by the company Cognis, oxyethylenated (5 EO) lauramide monosulphosuccinate sold under the name Lebon A-5000® by the company Sanyo.

As alkyl sulphoacetates, mention may, for example, be made of the mixture of sodium lauryl sulphoacetate and disodium lauryl ether sulphosuccinate sold under the name Stepan-Mild LSB by the company Stepan.

As polypeptides, mention may, for example, be made of those obtained by condensation of a fatty chain with amino acids from wheat and from oats, such as the potassium salt of hydrolysed lauroyl wheat protein sold under the name Aminofoam W OR® by the company Croda, the hydrolysed cocoyl soya protein, triethanolamine salt, sold under the name May-Tein SY® by the company Maybrook, the sodium salt of lauroyl oat amino acids sold under the name Proteol Oat® by the company Seppic, the hydrolysate of collagen grafted onto coconut fatty acids sold under the name Geliderm 3000® by the company Deutsche Gelatine, the soybean proteins acylated with hydrogenated coconut acids, sold under the name Proteol VS 22® by the company Seppic.

As anionic derivatives of alkyl polyglucoside, mention may be made of citrates, tartrates, sulphosuccinates, carbonates and glycerol ethers, produced from alkylpolyglucosides, such as the sodium salt of cocoyl polyglucoside (1.4) tartaric ester, sold under the name Eucarol AGE-ET® by the company Cesalpinia, the disodium salt of cocoyl polyglucoside (1.4) sulphosuccinic ester, sold under the name Essai 512 MP® by the company Seppic, the sodium salt of cocoyl polyglucoside (1.4) (1.4) citric ester, sold under the name Eucarol AGE-EC® by the company Cesalpinia, and the sodium lauryl polyglucoside ether carboxylate sold under the name Plantapon LGC Sorb by the company Cognis.

According to a particular embodiment of the invention, the surfactant system comprises, besides the surfactants of alkyl glycol carboxylate type and of alkyl phosphate type, at least one non-ionic surfactant, in particular chosen from alkylpolyglucosides.

Additives

The composition of the invention can contain any additives or active agents used in cleansing products. Mention may, for example, be made of preserving agents; sequestering agents (EDTA); antioxidants; fragrances; dyestuffs such as soluble dyes and pigments; pearlescent agents; mattifying, bleaching or exfoliating, mineral or organic fillers, providing viscosity; sunscreens; hydrophilic or lipophilic, cosmetic or dermatological active agents, such as water-soluble or liposoluble vitamins, antiseptics, antiseborrheic agents, antimicrobial agents such as benzoyl peroxide, salicylic acid, triclosan, azelaic acid or niacinamide (vitamin PP), and also optical brighteners; non-ionic, anionic, cationic and/or amphoteric polymers; fatty substances incompatible with aqueous medium, such as oils or waxes; viscosity modifiers or thickeners, or other agents having the effect of improving the cosmetic properties of the hair or of the skin. The amounts of these various adjuvants are those typically used in the field under consideration, and are, for example, from 0.01 to 20% of the total weight of the composition. These adjuvants and the concentrations thereof should be such that they do not modify the property desired for the composition of the invention.

The composition of the invention may in particular contain viscosity modifiers and thickeners, the amount of which depends on the viscosity desired for the final composition. These agents can be present at concentrations preferably ranging from 0.05 to 10% by weight, and preferably ranging from 0.05 to 5% by weight, relative to the total weight of the composition.

As viscosity modifiers and thickeners, mention may be made of electrolytes; scleroglucans; thickening polymers; particles; fatty amides; and mixtures thereof.

The thickening polymers may be anionic, amphoteric, cationic or non-ionic, may or may not be crosslinked, may or may not be hydrophobic modified polymers, and may be natural or synthetic.

As thickening polymers, use may, for example, be made of polymers derived from carboxylic acid, from polyacrylamide and/or from 2-acrylamidomethylpropane-sulphonic acid. The derived carboxylic polymer(s) may be associative polymers (i.e. containing a hydrophobic group) or non-associative polymers, that are water-soluble or water-dispersible, and that may or may not swell in an alkaline medium. They may be in the form of powder, of latex, or of an emulsion, or may be dispersed in water. The polymers may be non-ionic, anionic, cationic, zwitterionic or amphoteric. The monomers present in the polymers are preferably chosen from styrene, butadiene, ethylene, acrylonitrile, chloroprene, vinylidene chloride, isoprene, isobutylene or vinyl chloride monomers, and esters of acrylic acid, methacrylic acid, vinylacetic acid, maleic acid, crotonic acid and itaconic acid. These monomers can be used alone or in combination or can be mixed with one or more ionic monomers, for instance acrylic or methacrylic acids in charged form.

The preferred anionic polymers contain a monomer derived from acrylic or methacrylic acid and are partially neutralized, for example the polymers sold under the names Carbopol 981 and Carbopol 1382 by the company Noveon, and the polymer sold under the name Acrysol 22 by the company R6hm & Haas.

As non-ionic thickening polymers, use may be made for example of oxyalkylenated derivatives of fatty acid esters or of fatty alcohol ethers, or polysaccharides such as xanthan gum. As oxyalkylenated derivatives of fatty acid esters or of fatty alcohol ethers, mention may in particular be made of ethoxylated alkyl or acyl derivatives of polyols, which may in particular be oxyethylenated derivatives of C₆-C₃₀ fatty acid esters or of C₆-C₃₀ fatty alcohol ethers, and of polyols such as glycerol, sorbitol, glucose or pentaerythritol, these oxyethylenated derivatives generally containing from 50 to 500 oxyethylenated groups, and preferably from 100 to 300 oxyethylenated groups. As compounds of this type, mention may, for example, be made of oxyethylenated (200 EO) glyceryl stearate, such as the product sold under the name Simulsol 220 TM® by the company Seppic, oxyethylenated (150 EO) pentaerythrityl tetrastearate, such as the product sold under the name Crothix® by the company Croda, oxyethylenated (120 EO) methyl glucose dioleate, such as the product sold under the name Glucamate DOE-120 Vegetal® by the company Amerchol, oxyethylenated (160 EO) sorbitan triisostearate, such as the product sold under the name Rheodol TW IS399C by the company Kao Chemicals, and mixtures thereof. As polysaccharides, mention may be made of cetyl hydroxyethylcellulose sold under the name Natrosol plus grade 330 CS by the company Hercules, and mixtures thereof.

Preferably, the thickening polymers are chosen from non-ionic and anionic polymers.

The composition according to the invention is preferably free of cationic polysaccharides such as guar gums or of cationic galactomannans. However, it may contain cationic polymers other than cationic polysaccharides, for instance those derived from acrylamide, from acrylate or from dimethyldiallylammonium chloride.

As formulation-thickening particles, use may be made of clays, for instance hectorites such as the Bentone MA sold by the company Elementis Specialities.

As fatty amides, use may, for example, be made of cocamide MEA and cocamide MIPA.

As electrolytes, use may, for example, be made of sodium chloride, potassium chloride and analogous salts.

The compositions according to the invention may have an appearance ranging from a fluid product to a gel. They are stable and have very good rinsability. They can constitute, for example, a product for cleansing and/or for removing makeup from the skin, the scalp and/or the hair, a scrubbing product and/or an exfoliant product for the skin. They may constitute more particularly a cleansing composition for the skin.

Another subject of the invention is the cosmetic use of the composition as defined above, as a product for cleansing and/or for removing makeup from the skin, the scalp and/or the hair, and/or as a scrubbing product and/or an exfoliant product for the skin.

The compositions according to the invention are preferably used in two ways:
spreading the gel in the hands, applying it to the face or the body and then massaging it in the presence of water so as to develop the foam directly on the face or the body;
developing the foam in the palm of the hands before applying it to the face or the body.

In the two cases, the foam is usually then rinsed off.

The compositions according to the invention can also constitute a composition for treating greasy skin, in particular when they contain a specific active agent for treating greasy skin, such as, for example, salicylic acid, azelaic acid, triclosan, piroctone olamine or niacinamide (vitamin PP).

Another subject of the invention is the use of the composition as defined above, for preparing a composition intended for treating greasy skin.

The examples that follow serve to illustrate the invention without, however, being limiting in nature. The amounts indicated are given as % by weight unless otherwise mentioned.

In the table below, all the percentages are expressed by weight of active material (AM).

Sensory Performance Levels:

The sensory performance levels of the compositions (foam qualities) were determined according to the protocol described below:

Before any use of the product, the hands are washed with household soap and then suitably rinsed and dried. Next, the protocol followed is as follows:

1—wet the hands by passing them under running water, and shake them three times to drain them,

2—place 1 g of product in the palm of one of the hands,

3—work the product between the two palms for 10 seconds,

4—add 2 ml of water and work the product again for 10 seconds,

5—add 2 ml of water and work the product again for 10 seconds,

6—evaluate the quality of the foam according to the criteria defined below,

7—rinse the hands under water,

8—wipe them.

The foam criteria are graded on a scale of 0 to 10.

steps 4-6: evaluation of the foam quality

the foam volume: the grade attributed is higher, the greater the volume.

the size of the bubbles making up the foam: the grade attributed is higher, the larger the bubbles.

the hold of the foam: the grade attributed is higher, the more elastic the foam and the less it runs.

step 7: evaluation during rinsing out

rinsing out: the grade attributed is lower, the greater the presence of a slippery film that is difficult to remove, which means that the grade is higher, the more easy it is to rinse out the product without leaving any film.

The evaluation panel consisted of 4 trained experts. The mean of the four grades makes it possible to compare the compositions according to each of the criteria.

### EXAMPLES 1 AND 2 ACCORDING TO THE INVENTION AND COMPARATIVE EXAMPLES 1 AND 2

| | Example 1 | Example 2 | | |
|---|---|---|---|---|
| | according | according | Comparative | Comparative |
| | to the | to the | example | Example |
| Composition | invention | invention | 1 | 2 |
| Sodium lauryl | 7.8 | 11.7 | 15.6 | 7.8 |
| glycol | | | | |
| carboxylate (1) | | | | |
| Monoalkyl | 6.5 | 3.25 | — | |
| phosphate (2) | | | | |
| Sodium laureth | — | — | — | 6.51 |
| sulphate (3) | | | | |
| Potassium | 1.7 | 0.85 | — | |
| hydroxide | | | | |
| Citric acid | — | — | 0.035 | |
| Imidazolidinyl | — | — | 0.2 | |
| urea | | | | |
| Water | qs 100 | qs 100 | qs 100 | |
| pH | 7.2 | 7 | 7.35 | 7 |
| Bubble size | 3.6 | 4 | 4.8 | 5 |
| Foam volume | 6.8 | 6.8 | 6.9 | 6.8 |
| Foam hold | 7.3 | 7.5 | 7.5 | 7.8 |
| Rinsing out | 9.1 | 9 | 8.5 | 8 |

(1) Beaulight Shaa (Sanyo) containing 30% of active material, introduced in an appropriate amount so as to have the % of active material indicated in the table.
(2) MAP 20 (KAO) (100% of active material)
(3) Empicol ESB 70/FL 2 (Huntsman) containing 70% of active material, introduced in an appropriate amount so as to have the % of active material indicated in the table.

The Examples and Comparative Examples show that the lauryl glycol carboxylate/alkyl phosphate combination provides foams that are finer while at the same time keeping a good foam volume with a high density, and also good rinsing out. On the other hand, the combination of lauryl glycol carboxylate with another anionic surfactant such as lauryl ether sulphate does not make it possible to reduce the size of the bubbles, as shown in Comparative Example 2.

### EXAMPLE 3 ACCORDING TO THE INVENTION AND COMPARATIVE EXAMPLE 3

| | Example 3 | |
|---|---|---|
| | according | Comparative |
| | to the | Example |
| Composition | invention | 3 |
| Sodium lauryl glycol | 5.2 | 7.8 |
| carboxylate (1) | | |
| Monoalkyl phosphate (2) | 4.33 | — |
| Sodium laureth sulphate (3) | — | — |
| Decyl glucoside (4) | 4.33 | 6.5 |
| Potassium hydroxide | 1.13 | — |
| Citric acid | — | 0.02 |
| Imidazolidinyl urea | — | — |
| Water | qs 100 | qs 100 |
| pH | 6.85 | 7.23 |
| Bubble size | 3.9 | 5.4 |
| Foam volume | 5.4 | 8.4 |
| Foam hold | 7.1 | 8.3 |
| Rinsing out | 9 | 8.9 |

(1) Beaulight Shaa (Sanyo) containing 30% of active material, introduced in an appropriate amount so as to have the % of active material indicated in the table.
(2) MAP 20 (KAO) (100% of active material)
(3) Empicol ESB 70/FL 2 (Huntsman) containing 70% of active material, introduced in an appropriate amount so as to have the % of active material indicated in the table.
(4) Mydol 10 (Kao) containing 40% of active material, introduced in an appropriate amount so as to have the % of active material indicated in the table.

This Example and Comparative Example show that the lauryl glycol carboxylate/alkyl phosphate combination provides foams that are finer while at the same time keeping good rinsing out. The above written description of the invention provides a manner and process of making and using it such that any person skilled in this art is enabled to make and use the same, this enablement being provided in particular for the subject matter of the appended claims, which make up a part of the original description and including a cleansing composition for topical application, comprising, in a physiologically acceptable aqueous medium, a surfactant system containing at least one surfactant chosen from alkyl glycol carboxylic acids and their salts, and at least one surfactant chosen from monoalkyl phosphates, dialkyl phosphates and their salts.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out. Terms such as “contain(s)” and the like as used herein are open terms meaning ‘including at least’ unless otherwise specifically noted.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in a physiologically acceptable aqueous medium, a surfactant system comprising at least one surfactant chosen from alkyl glycol carboxylic acids and their salts, and at least one surfactant chosen from monoalkyl phosphates, dialkyl phosphates, and their salts.

2. The composition according to claim 1, wherein the surfactant system is present in an amount ranging from 2 to 30% by weight relative to the total weight of the composition.

3. The composition according to claim 1, wherein the weight ratio of the surfactants chosen from alkyl glycol carboxylic acids and their salts to the surfactants chosen from monoalkyl phosphates, dialkyl phosphates, and their salts is 90/10 to 10/90.

4. The composition according to claim 1, comprising at least one surfactant of formula (I) below:
R₁—CHOH—CH₂—O—CH₂—COO⁻X⁺ (I)
in which R₁ denotes a linear or branched, saturated or unsaturated alkyl radical containing from 8 to 30 carbon atoms, and X denotes hydrogen or a mineral or organic cation.

5. The composition according to claim 4, wherein R₁ denotes a linear or branched, saturated or unsaturated alkyl radical containing from 8 to 18 carbon atoms.

6. The composition according to claim 1, comprising at least one of sodium lauryl glycol carboxylate and lauryl glycol carboxylic acid.

7. The composition according to claim 1, wherein the amount of surfactant(s) chosen from alkyl glycol carboxylic acids and their salts ranges from 1 to 20% by weight relative to the total weight of the composition.

8. The composition according to claim 1, wherein the surfactant chosen from monoalkyl phosphates, dialkyl phosphates, and their salts is chosen from lauryl monophosphate, the potassium salt of dodecylphosphoric acid, the mixture of monoester and diester of octylphosphoric acid, the mixture of monoester and diester of ethoxylated (7 mol of EO) 2-butyloctanyl phosphoric acid, the potassium or triethanolamine salt of (C₁₂-C₁₃) monoalkyl phosphate, potassium lauryl phosphate, and mixtures thereof.

9. The composition according to claim 1, wherein the amount of surfactant(s) chosen from monoalkyl phosphates, dialkyl phosphates, and their salts ranges from 0.5 to 10% by weight relative to the total weight of the composition.

10. The composition according to claim 1, wherein the surfactant system further comprises one or more additional non-ionic, amphoteric or anionic surfactants.

11. The composition according to claim 10, comprising at least one alkylpolyglucoside.

12. The composition according to claim 1, further comprising a thickener chosen from thickening polymers, particles, fatty amides, electrolytes, and mixtures thereof.

13. The composition according to claim 1, wherein the weight ratio of the surfactants chosen from alkyl glycol carboxylic acids and their salts to the surfactants chosen from monoalkyl phosphates, dialkyl phosphates, and their salts is 25/75 to 75/25.

14. The composition according to claim 1, wherein said composition is a scrubbing product and/or an exfoliant product for the skin.

15. A method, comprising applying the composition of claim 1 to the skin, the scalp and/or the hair.

16. The method according to claim 15, wherein said method is a method for cleansing and/or for removing makeup from the skin, the scalp and/or the hair.

17. The method according to claim 15, wherein said method is a method for scrubbing or exfoliating the skin.

18. The method according to claim 15, wherein the composition is spread in the hands, applied to the face or the body and then massaged in the presence of water so as to develop a foam directly on the face or the body.

19. The method according to claim 15, wherein the composition is spread in the hands, applied to the face or the body and then massaged in the presence of water so as to develop a foam directly on the face or the body.
